(19) United States
(12) Patent Application Publication
(10) Pub. No.: US 2024/0299716 A1
(43) Pub. Date: Sep. 12, 2024

(54) SHOWER THERAPY AND FILTER DEVICE

(71) Applicants: Michelle Vidal, Valencia, CA (US); Freddy Vidal, Valencia, CA (US)

(72) Inventors: Michelle Vidal, Valencia, CA (US); Freddy Vidal, Valencia, CA (US)

(21) Appl. No.: 18/599,725

(22) Filed: Mar. 8, 2024

Related U.S. Application Data
(60) Provisional application No. 63/450,821, filed on Mar. 8, 2023.

(51) Int. Cl. A61M 35/00; A61M 21/00; A61M 21/02; C02F 1/00; C02F 1/68; C02F 5/14; C02F 9/20; C02F 101/10; C02F 101/20; C02F 103/42; E03C 1/26

(52) U.S. Cl. CPC A61M 35/20; A61M 21/02; C02F 9/20; E03C 1/26; A61M 2021/0016; A61M 2205/75; C02F 1/003; C02F 1/68; C02F 5/14; C02F 2101/101; C02F 2101/203; C02F 2103/42; C02F 2307/06

(57) ABSTRACT

The present invention discloses a shower therapy device that is designed to be affixed inline and between a shower arm and a showerhead of a shower. The shower therapy device provides filtered water along with aroma therapy and the provision of mineral treated water. The shower therapy device includes a junction component that is detachably attached to a housing. The shower therapy device includes a fragrance pod in the junction component that provides aromatic therapeutic to the user, in addition to filtering action provided by filters accommodated in the housing.

### CROSS-REFERENCE TO RELATED APPLICATIONS

The present application is a non-provisional application of U.S. Provisional Application No. 63/450,821, filed Mar. 8, 2023, the entirety of which is incorporated herein by reference.

### FIELD OF THE INVENTION

The present invention relates to a shower accessory and, more particularly, to a shower filter and therapy device that filters shower water and provides an aromatic therapeutic effect to users.

### BACKGROUND OF THE INVENTION

A shower head is a device that is generally used for discharging cold or hot water at a certain pressure within a shower. The shower head is connected to a faucet and is usually used with tap water. Tap water discharged through the shower head can contain harmful substances such as rust caused by the corrosion of the water pipe, heavy metals due to contamination of the water, and harmful substances such as chlorine administered during the purification process of the tap water, which can cause skin troubles, etc. In some instances, tap water has been reported to have several side effects. In general, showers and associated shower heads are not provided with a filter, so a user is bound to use unclean or otherwise unfiltered water.

To reduce the harmful substances and contamination from the tap water, a shower head with filter assemblies are available in the market. In addition to this a variety of shower filters, such as mesh screens or cartridges have long been known in the art and are typically used to remove particles before entering the shower. The shower filters are usually mounted at the water inlet end, while tap water is filtered through mesh screens or cartridges in an attempt to remove contaminants or impurities from the tap water. A few examples of such types of shower filters are explained below.

A U.S. Pat. No. 6,267,887, assigned to 3M Innovative Properties Co., describes multi-purpose, replaceable shower or tub filter cartridges that are integrated with a shower. The shower filter attempts to remove chlorine and treat components that causes scaling in the shower environment.

A U.S. Pat. No. 6,270,023, assigned to David K. Farley, describes a combination of a hand-held shower head and a filter assembly to remove chlorine and other substances from water. This assembly includes a two-stage filter media of (1) ceramic-type calcium sulfite beads and (2) a copper/zinc material, both held in an internal chamber formed in an elongated, hollow handle.

Another reference, U.S. Publication No. 2013/0319929, assigned to David Aaron Farley, discloses a canister style shower filter assembly that is disposed between the shower arm and the showerhead. The shower filter assembly includes a cap having an input port connectable to the shower arm and an output port connectable to a showerhead, a filter assembly engageable to and supportable by the cap, and a housing disposed about the filter assembly which is separately engageable to the cap.

Although existing shower assemblies with integrated filter cartridges or filter screens provide a basic filtering effect, they are comprised of complex structures and, as a result, are difficult to assemble and disassemble. Further, existing shower filter assemblies fail to provide any enhanced or therapeutic effect.

Thus, a continuing need exists for a shower accessory that provides a filtering function to filter and/or treat the tap water as well as a therapeutic aroma to the user.

### SUMMARY OF THE INVENTION

In accordance with the present invention, the disadvantages and limitations of the prior art are substantially avoided by providing a canister style shower therapy device that is disposed between a shower arm and a showerhead of a shower. The shower accessory is affixed inline with a shower and effectively removes chlorine from tap water and treats components of the tap water, such as filtering fine sediment from the tap water and removing materials that cause scale deposits, etc.

The shower therapy device includes a junction component that further includes an input connectable to the shower arm for receiving untreated water and an outlet connectable to the showerhead for delivering treated water. The junction component is in form of a cap. The junction component includes a top portion with a recess or cavity and a central portion is protruded out from bottom of the top portion. The central portion is connected to the outlet for delivering treated water.

The shower therapy device includes a fragrance pod unit for providing therapeutic effect to a user. The fragrance pod unit includes a fragrance pod within the cavity of the top portion of the junction component. A fragrance pod cap including one or more holes is positioned to cover the cavity to allow passage of aroma from the fragrance pod, further wherein the fragrance pod cap covers the fragrance pod. The cavity includes a plurality of walls and further the plurality of walls are heated by a warm water that passes along the shower accessory or the shower therapy device and further passing heating effect to the fragrance pod positioned within the cavity to emit aroma The fragrance pod includes any desired fragrance material to provide a desired fragrance, non-limiting examples of which include liquid/oil or other material that provides a lavender fragrance, a eucalyptus fragrance or a lemon grass fragrance.

The shower therapy device includes a housing detachably attached to the junction component. The housing includes a hair and skin pod unit that includes a hair and skin pod with one or more substances. The one or more substances are selected from a list of hair solutions, skin solutions, hair powders or skin powders. The hair and skin pod receives the untreated water from the inlet through a first fluid flow path to mix the untreated water with the one or more substances to form an unfiltered water. The hair and skin pod unit includes a hair and skin pod cap to cover the hair and skin pod.

The housing further includes a filtering unit that receives the unfiltered water from the hair and skin pod cap to provide the treated water. Further, the treated water passes through a second fluid flow path via the central portion to deliver the treated water to the outlet.

A primary objective of the present invention is to provide a shower therapy device that can be used as an environment-friendly product by reducing environmental waste compared to existing products, and can function as a high-performance filter by increasing filter efficiency compared to the existing product.

Another objective of the present invention is to provide a shower therapy device that provides aromatic therapeutic effect to a user while showering in addition to filtering of the untreated water.

Yet another objective of the present invention is to provide a multi-purpose shower accessory that includes multiple filter cartridges or pods that is designed to reduce chlorine in tap water through adsorption to reduce or eliminate scale deposits.

In alternative embodiment of the present invention, a shower accessory including a junction component that includes an inlet for receiving an untreated water, a central portion, an outlet for delivering a treated water. The junction component includes a fragrance pod unit that includes a fragrance pod within a cavity of the fragrance pod unit, and a fragrance pod cap including one or more holes to allow passage of aroma from the fragrance pod, further wherein the fragrance pod cap covers the fragrance pod.

The shower accessory includes a housing detachably attached to the junction component. The housing includes a hair and skin pod unit that includes one or more of a hair substance and/or a skin substance, or any combination thereof, to provide therapeutic benefits to the skin and hair of a user. The hair and skin pod receives the untreated water from the inlet through a first fluid flow path to mix the untreated water with the one hair substance and/or the skin substance to form an unfiltered water with hair/skin substances. A hair and skin pod cap is used to cover the hair and skin pod, wherein the hair and skin pod cap includes a mesh screen. The housing includes a filtering unit that receives the unfiltered water from the mesh screen. The filtering unit includes any desirable filter components, non-limiting examples of which include a layer of carbon, calcium sulfite and KDF 85 and a crystal quartz layer on top of the layer of carbon. The unfiltered water passes through the filtering unit to remove iron and hydrogen sulfide and/or other components from the unfiltered water to produce the treated water. The treated water passes through a second fluid flow path via the central portion to deliver the treated water through the outlet.

Other objectives and aspects of the invention will become apparent from the following detailed description, taken in conjunction with the accompanying drawings, which illustrate, by way for example, the features in accordance with embodiments of the invention.

To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims.

Although, the invention is described above in terms of various exemplary embodiments and implementations, it should be understood that the various features, aspects, and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

The presence of broadening words and phrases such as “one or more,” “at least,” “but not limited to” or other like phrases in some instances shall not be read to mean that the narrower case is intended or required in instances where such broadening phrases may be absent.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles. Furthermore, the drawings may contain text or captions that may explain certain embodiments of the present invention. This text is included for illustrative, non-limiting, explanatory purposes of certain embodiments detailed in the present invention. In the drawings:

Embodiments of the invention are described with reference to the following figures. The same numbers are used throughout the figures to reference like features and components. The features depicted in the figures are not necessarily shown to scale. Certain features of the embodiments may be shown exaggerated in scale or in somewhat schematic form, and some details of elements may not be shown in the interest of clarity and conciseness.

FIG. 1 illustrates a shower therapy device in closed configuration in accordance with the present invention;

FIG. 2 illustrates the shower therapy device in open configuration in accordance with the present invention;

FIG. 3 illustrates a perspective view of the shower therapy device in open configuration focusing on attachment of a junction component and a housing in accordance with the present invention;

FIG. 4 illustrates a perspective view of a housing focusing on a hair and skin pod unit and a filtering unit of the shower therapy device in accordance with the present invention;

FIG. 5 illustrates a cross-sectional view of the shower therapy device focusing on the flow of water through the shower therapy device in accordance with the present invention;

FIG. 6 illustrates a cross-sectional view of the shower therapy device focusing on attachment of the junction component and the housing components in accordance with the present invention;

FIG. 7 illustrates detailed view of the housing components of the shower therapy device in accordance with the present invention; and

FIG. 8 illustrates a detailed view of the shower therapy device focusing on dimension of the junction component and the housing in accordance with the present invention.

### DETAILED DESCRIPTION OF THE DRAWINGS

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “units” represents the dimension in any units such as centimeters, meters, inches, foots, millimeters, micrometer and the like and forms thereof, are not necessarily limited to members in a list with which the words may be associated.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

Regarding applicability of 35 U.S.C. § 112, 16, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items from the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present invention contains headers. It should be understood that these headers are used as references and are not to be construed as limiting upon the subjected matter disclosed under the header.

This specification includes references to “one embodiment” or “an embodiment.” The appearances of the phrases “in one embodiment” or “in an embodiment” do not necessarily refer to the same embodiment. Particular features, structures, or characteristics may be combined in any suitable manner consistent with this disclosure.

The presence of broadening words and phrases such as “one or more,” “at least,” “but not limited to” or other like phrases in some instances shall not be read to mean that the narrower case is intended or required in instances where such broadening phrases may be absent.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

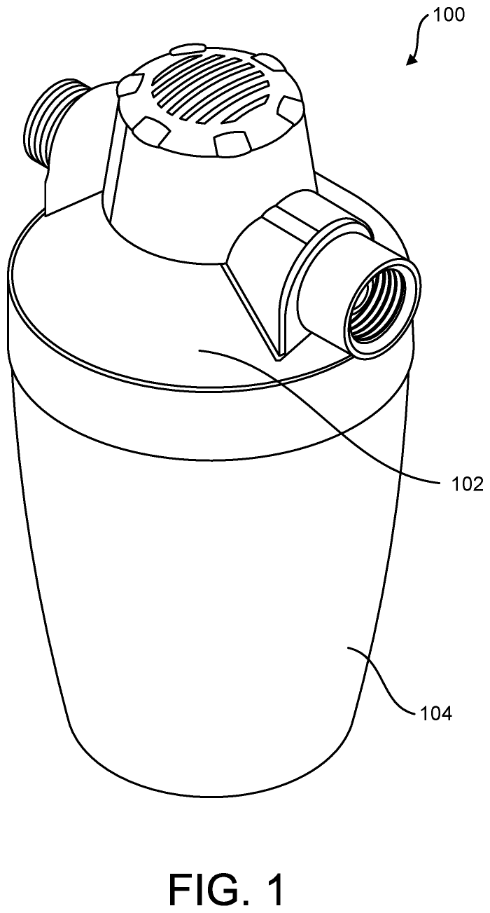

FIG. 1 illustrates a shower therapy device (100) in closed configuration. The shower therapy device includes a junction component (102) and a housing (104) that is detachably attached to the junction component (102). In one aspect, the shower therapy device (100) is a canister style shower therapy device that is disposed between a shower arm and a showerhead of the shower. The junction component (102) is an upper part of the shower therapy device (100) in form of a cap that provides a coupling point for multiple units.

The junction component (102) includes an inlet connectable to the shower arm for receiving untreated water. The untreated water is, for example, tap water and either cold water or hot water, or any combination thereof. The junction component (102) includes an outlet connectable to the showerhead for delivering treated water. Further, the junction component (102) includes a top portion with a recess or a cavity. Further, the junction component includes a central portion that is further connected to the outlet and protrudes out below the central portion.

The junction component (102) includes a fragrance pod unit that includes a fragrance pod positioned within a cavity and a fragrance pod cap to covers the cavity. The cavity is surrounded by walls that get heated by the treated water to allow emission of aroma from the fragrance pod. The fragrance pod includes any material that emits a desired fragrance, non-limiting examples of which include a scented liquid or oil, such as lavender oil, lemongrass oil, or eucalyptus oil. The walls of the cavity is heated by warm water that passes through the shower therapy device and passes the heating effect through convection or conduction heating to the fragrance pod to release fragrance. The outlet delivers treated water.

The housing (104) is detachably attached to bottom of the junction component of the shower therapy device. The housing (104) includes a hair and skin pod unit and a filtering unit. The hair and skin pod unit includes a hair and skin pod and hair and skin pod cap. The hair and skin pod includes one or more substances that treat hair and skin along with the water as it passes through the housing. The hair and skin pod cap covers the hair and skin pod. The housing includes a filtering unit affixed to the hair and skin pod unit. The filtering unit includes components to remove chlorine and volatile organic compounds (VOCs) in the water, further where the filter sends the treated water to the outlet unit.

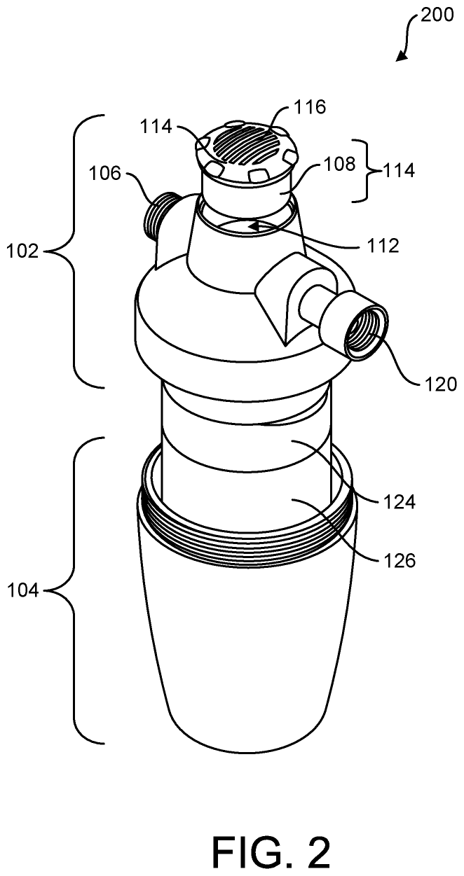

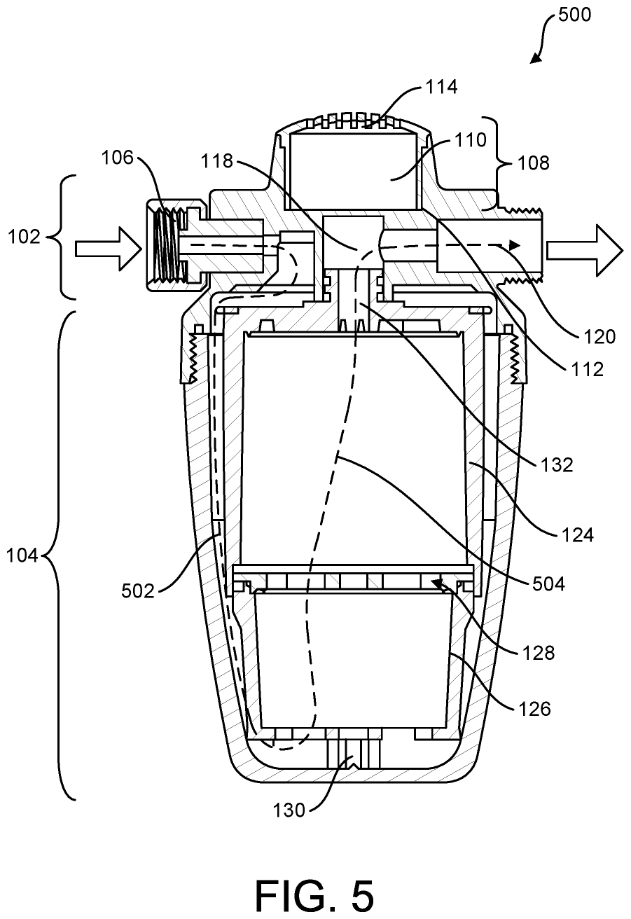

FIG. 2 illustrates a front view of the shower therapy device (100) in an open configuration. The shower therapy device (100) includes a junction component (102) and a housing component (104). The junction component (102) includes an inlet (106) for receiving the untreated water. The junction component (102) includes a top portion with a recess or a cavity (112). The junction component (102) includes a fragrance pod unit (108) for delivering aromatic effect to the user. The fragrance pod unit (108) includes a fragrance pod (110) within a cavity (112). The fragrance pod unit (108) includes a fragrance pod cover (114) for distributing aroma to the user. Further, the fragrance pod cover (114) includes one or more holes (116) to allow passage of aroma from the fragrance pod (110). Further, the junction component (102) includes a central portion (118) (shown in FIG. 5) fluidly connected to an outlet (120) for delivering treated water.

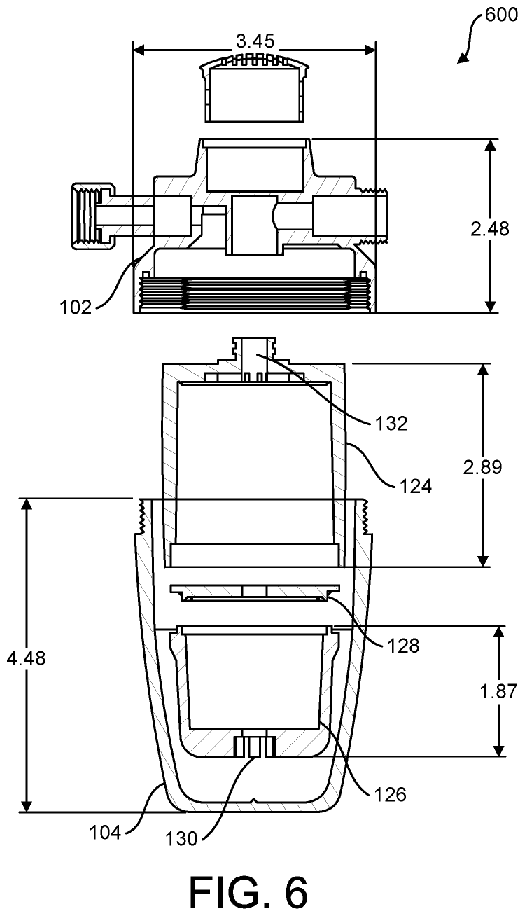

Further, the housing (104) includes a hair and skin pod (126) and a filtering unit (124). The hair and skin pod (126) includes a hair and skin pod cap (128) (shown in FIGS. 5 through 6). The hair and skin pod (126) includes one or more substances to treat the untreated water. In alternative embodiment of the present invention, the hair and skin pod (126) includes at least one hair substance and at least one skin substance. Further, the hair and skin pod (126) receives the untreated water via a first port (130) from the inlet (106) through a first fluid flow path to mix the untreated water with the one or more substances to form an unfiltered water. The hair and skin pod cap (128) is used to cover the hair and skin pod (126) components and, in some aspects, includes a mesh screen allowing passage of unfiltered water.

Further, the filtering unit (124) receives the unfiltered water from the mesh screen (128) to remove impurities or contaminates to treat the water and produce treated water. In one embodiment of the present invention, the filtering unit (124) includes a layer of carbon, calcium sulfite and KDF 85 and a crystal quartz layer on top of the layer of carbon or any other layer, or the other way round or any combination thereof. The unfiltered water passes through the filtering unit to remove iron and hydrogen sulfide from the unfiltered water to produce the treated water. The treated water passes through a second fluid flow path from a second port (132) via the central portion to deliver the treated water through the outlet (120).

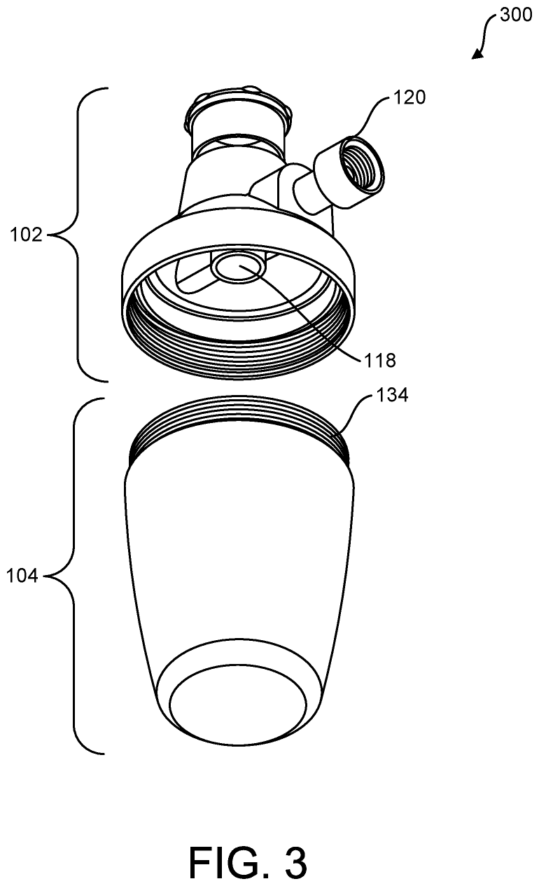

FIG. 3 illustrates perspective view of the shower therapy device (100) in open configuration focusing on the central portion (118). The shower therapy device (100) as can be seen in the open configuration includes a junction component (102) and housing (104). The junction component includes a central portion (118) that is designed to receive and seal against the second port (shown as element 132 in FIG. 6) on the filtering unit. The second port 132 can be screwed into the central portion (118) or, in another aspect, can include O-rings for sealing and press fit into the central portion (118). The central portion (118) is connected to the outlet (120) to deliver the treated water from the filtering unit of the housing (104).

The junction component (102) comprises of the upper portion of the shower therapy device (100) from where the untreated water enters, and further water treated within the housing (104) and freshly filtered and treated water is poured out from the outlet (120). The junction component (102) is detachably connected to the housing (104) of the shower therapy device. In one aspect, the junction component (102) is threadably engaged to the housing (104). The housing (104) includes a plurality of threads (134) to engage the junction component (102) and allow a user to selectively open the housing (104) to replace the components therein. In an alternative embodiment of the present invention, the junction component (102) is attached to the housing (104) through a screw type engagement or snap fit type engagement or any other suitable engagement as understood by those skilled in the art.

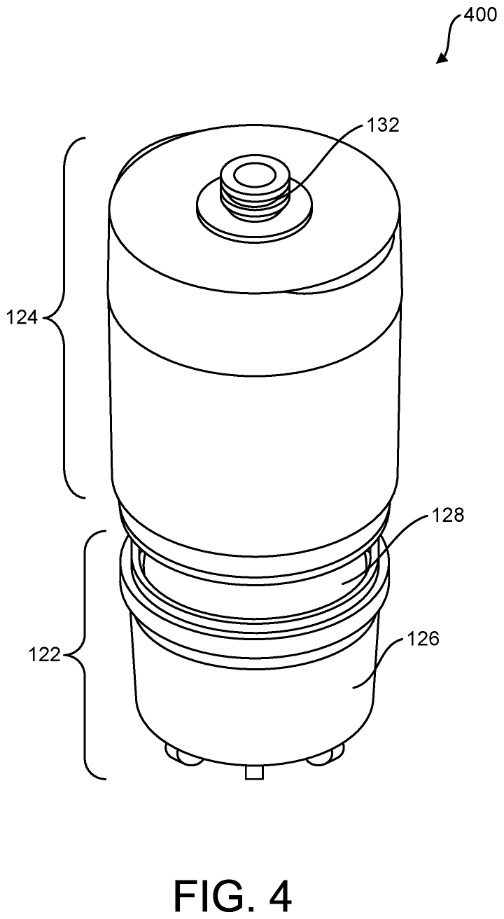

FIG. 4 illustrates a perspective view of the hair and skin pod (126) and the filtering unit (124) of the shower therapy device (100), focusing on the ports for transporting water (400). As noted herein, the hair and skin pod (126) includes a first port for receiving the untreated water via a first fluid flow path from the inlet (106). The filtering unit (124) includes a second port (132) that is used to attach to the junction component (102). In a preferred embodiment of the present invention and as noted above, the second port (132) is screwed or otherwise affixed to the junction component through the central portion (e.g., being press fit with O-rings). The second port (132) receives the treated water from the filtering unit and transfers the treated water via a second fluid flow path from the outlet of the junction component.

The hair and skin pod (126) is affixed to the filtering unit (124). In a preferred embodiment of the present invention, the hair and skin pod unit is affixed through any attachment means known to those skilled in the art. In a preferred embodiment of the present invention, the hair and skin pod (126) is threadly engaged to the filtering unit (124). The housing (104) further encases and secures both the filtering unit (124) and the hair/skin pod (126). The filtering unit (124) includes any desired components to treat and filter the water passing there through.

As a non-limiting example, the filter has one or more screens and can also include components to remove chlorine and volatile organic compounds (VOCs) in the water. In a preferred embodiment of the present invention, the filtering unit (124) includes a layer of filters, such as crystal quartz on top, then carbon+calcium sulfite+KDF 85 that effectively removes iron and hydrogen sulfide from water. Although one order of layers is described, it should be understood that the order of the layering can be altered as desired.

Additionally, the hair and skin pod (126) is formed to house one or more components/ingredients to treat the water as it passes therethrough. Any desired component/ingredient known in the art can be included to treat the water. In one embodiment of the present invention, the hair and skin pod (126) includes one or more substances for dry skin and hair. In a desired embodiment of the present invention, the hair and skin pod (126) includes zeolites and a softening resin compositition.

As a non-limiting example, the softening resin is Siliphos which is a natural polyphosphate mineral extracted from the milk thistle seed. Siliphos is a widely-trusted, FDA approved, food-grade mineral. Thus, as applied to the present invention, Siliphos is a water treatment mineral compound, a crystal Polyphosphate containing Sodium, Calcium Magnesium and Silicate, formulated specifically for the treatment of hard water problems.

In another embodiment of the present invention, the hair and skin pod (126) includes one or more substances for oily hair/skin. The hair and skin pod (126) for oily hair/skin includes carbon and an oil control tablets composition. In a preferred embodiment of the present invention, the carbon is used as carbon ceramic beads, while the oil control tablets include one or more ingredients that control the production of oily hair and skin. Examples of the one or more ingredients includes Dium Cocoyl Glutamate 68187-32-6 31, Methyl Coconut Oil Acyl Sodium Taurine 12765-39-8 30, Sodium Lauroyl Isethionate 7381-01-3 25, Aqua 7732-18-5 4.28, Hydrolyzed Corn Starch 9005-25-8 3, Glycerin 56-81-5 2, Sodium Lauryl Sulfate 151-21-3 2, Fragrance N.A. 1.5, Olea Europaea (Olive) Fruit Oil 8001-25-0 1, Allantoin 97-59-6 0.1, Piroctone Olamine 68890-66-4 0.1, Titanium Dioxide 13463-67-7 0.02 100 and any combination thereof.

In one another embodiment of the present invention, the hair and skin pod (126) includes one or more substances for dandruff. The hair and skin pod (126) for dandruff includes an anti-dandruff material and zeolites composition. The anti-dandruff materials is one or more items selected from a list of ingredients that include Odium Cocoyl Glutamate 68187-32-6 31, Methyl Coconut Oil Acyl Sodium Taurine 12765-39-8 30, Sodium Lauroyl Isethionate 7381-01-3 25, Aqua 7732-18-5 4.28, Hydrolyzed Corn Starch 9005-25-8 3, Glycerin 56-81-5 2, Sodium Lauryl Sulfate 151-21-3 2, Fragrance N.A. 1.5, Olea Europaea (Olive) Fruit Oil 8001-25-0 1, Allantoin 97-59-6 0.1, Piroctone Olamine 68890-66-4 0.1, Titanium Dioxide 13463-67-7 0.02 100, and any combination thereof.

In one embodiment of the present invention, the hair and skin pod (126) includes one or more substances for an anti-frizz treatment. In one example aspect, the anti-frizz treatment composition includes one or more far-infrared molecules and carbon and an anti-frizz formula. In an exemplary embodiment of the present invention, the hair and skin pod (126) include one ceramic bead with far infrared ingredients, such as Quartz and Hematite, which produces smaller water molecules.

The anti-frizz formula controls frizz in the hair and can be any suitable ingredient, such as one or more of chemical ingredient selected from a group consisting of (including the respective CAS No. and percentage % Content) Sodium Cocoyl Glutamate 68187-32-6 31, Methyl Coconut Oil Acyl Sodium Taurine 12765-39-8 30, Sodium Lauroyl Isethionate 2001899 25, Aqua 7732-18-5 4.18, Hydrolyzed Corn Starch 9005-25-8 3, Glycerin 56-81-5 2, Pyroxone Ethanolamine Salt N.A. 2, Fragrance N.A. 1.5, Olea Europaea (Olive) Fruit Oil 8001-25-0 1, Butyrospermum Parkii (Shea) Butter 194043-92-0 0.1, Macadamia Ternifolia Seed Oil 128497-20-1 0.1, Allantoin 97-59-6 0.1, and Titanium Dioxide 13463-67-7 0.02, or any combination thereof.

FIG. 5 illustrates a detailed view of the flow of water (500) through the shower therapy device. The flow of water (500) in the shower therapy device starts from the inlet (106) of the junction component (102), which receives untreated water. The untreated water is either cold water or warm water. In preferred embodiment of the present invention, the untreated water is warm water. As the warm water passes through the shower therapy device, it heats up the surrounding walls of the cavity and transfers the heating effect to the of the fragrance pod (110). The aroma from the fragrance pod (110) can then easily passes through one or more holes (116) from the fragrance pod cap (114).

The untreated water from the inlet (106) moves from one side wall of the housing (104) to a first port (130) at the bottom portion of the hair and skin pod (126). The hair and skin pod (126) receives the untreated water from the inlet (106) through a first fluid flow path (502) to mix the untreated water with or more substances, more specifically, at least one hair substance and/or the at least one skin substance to form an unfiltered water. The unfiltered water passes through a hair and skin pod cover (128) (i.e., a mesh screen) to the filtering unit (124).

The filtering unit (124) receives the unfiltered water from the mesh screen. The unfiltered water then passes through the filtering unit (124) to remove impurities and contaminants from the unfiltered water. More specifically and in one aspect, the filtering unit (124) removes iron and hydrogen sulfide from the unfiltered water to produce the treated water. The treated water then passes through a second fluid flow path (504) via the second port (132) to the central portion (118) to deliver the treated water through the outlet (120).

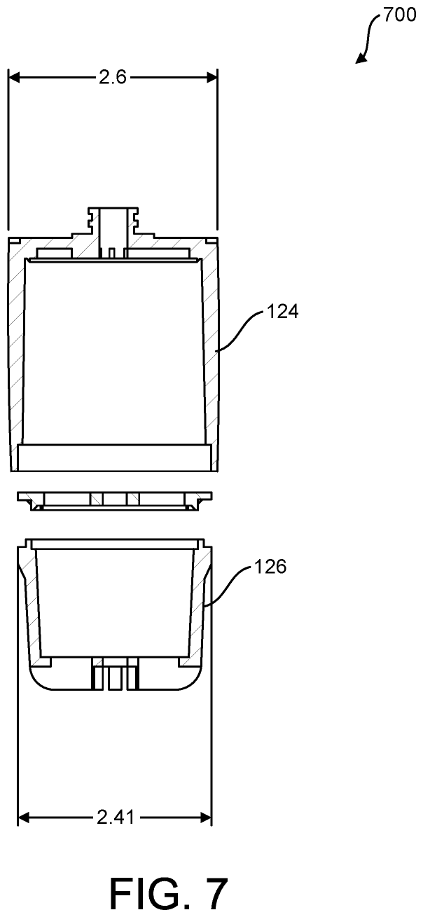

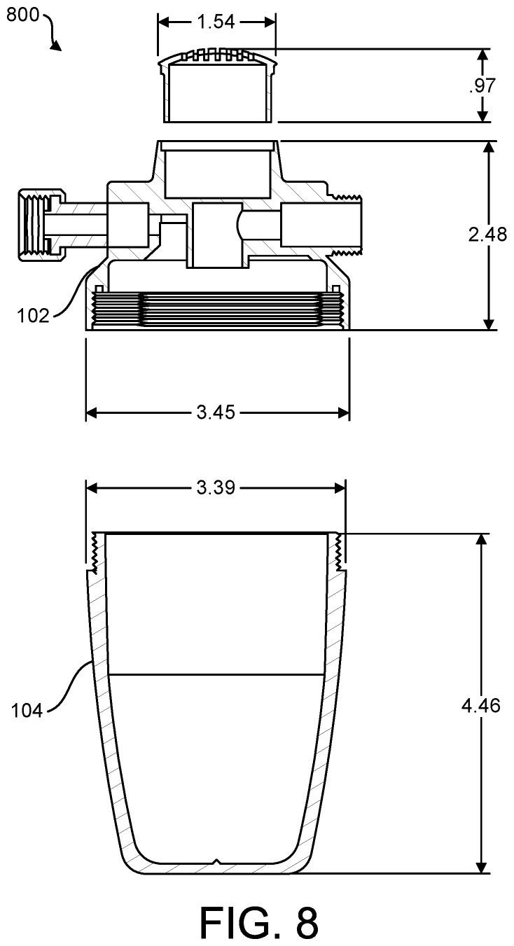

FIG. 6 illustrates a cross-sectional view of the shower therapy device (100), focusing on attachment of the junction component (102) and the housing (104) components in accordance with the present invention. Moreover, FIGS. 6-8 provide several non-limiting examples of suitable dimensions of each component of the shower therapy device in detail. It should be noted that although dimensions are provided for illustrative purposes, the invention is not intended to be limited thereto as other shapes and dimensions can be implemented in accordance with the present invention. As shown in FIG. 6, the dimension of the junction component (102) is represented as length and width. In this non-limiting example, the length of the junction component (102) is 2.48 units and the width of the junction component (102) is 3.45 units. In another aspect, the length of the housing is 4.48 units, while the length of the hair and skin pod (126) is 1.87 units and the length of the filtering unit (124) is 2.48 units. Further and as shown in FIG. 7, the width of the hair and skin pod (126) is 2.41 units, while the width of the filtering unit (124) is 2.6 units. Further and as shown in FIG. 8, the width of the fragrance pod cap (114) is 1.54 units, while the length of the fragrance pod cap (110) is 0.97 units and the length of the housing (104) is and measured as 4.46 units. Finally, the measurement of the female part in the housing (104) is 3.39 units and the male part of the junction component (102) is 3.45 units.

Finally, while this invention has been described in terms of several embodiments, one of ordinary skill in the art will readily recognize that the invention may have other applications in other environments. It should be noted that many embodiments and implementations are possible. Further, the following claims are in no way intended to limit the scope of the present invention to the specific embodiments described above. In addition, any recitation of “means for” is intended to evoke a means-plus-function reading of an element and a claim, whereas, any elements that do not specifically use the recitation “means for”, are not intended to be read as means-plus-function elements, even if the claim otherwise includes the word “means”. Further, while particular method steps have been recited in a particular order, the method steps may occur in any desired order and fall within the scope of the present invention.

1. A shower therapy device, comprising:
a junction component, wherein the junction component includes:
an inlet for receiving untreated water and directing the untreated water to a housing;
a central portion for receiving treated water;
an outlet in fluid communication with the central portion for delivering the treated water; and
a housing detachably attached to the junction component, the housing formed to accommodate a hair and skin pod and a filtering unit and direct water to the central portion.

2. The shower therapy device as set forth in claim 1, further comprising a hair and skin pod having one or more substances therein, wherein the hair and skin pod receives the untreated water from the inlet through a first fluid flow path to mix the untreated water with the one or more substances to form an unfiltered water.

3. The shower therapy device as set forth in claim 2, further comprising a filtering unit for attaching with and between the hair and skin pod and the central portion, the filtering unit adapted to receive the unfiltered water from the hair and skin pod to provide treated water and direct the treated water to the central portion, where it is delivered through the outlet.

4. The shower therapy device as set forth in claim 3, wherein the junction component further comprises a fragrance pod unit having a cavity for receiving a fragrance pod.

5. The shower therapy device as set forth in claim 4, wherein the cavity is surrounded by walls that get heated by the treated water to allow emission of aroma from the fragrance pod.

6. The shower therapy device as set forth in claim 4, wherein the fragrance pod includes a fragrance selected from a group consisting of a lavender fragrance, a eucalyptus fragrance, and a lemon grass fragrance.

7. The shower therapy device as set forth in claim 2, wherein the one or more substances are zeolites and softening resins.

8. The shower therapy device as set forth in claim 7, wherein the softening resins include Siliphos.

9. The shower therapy device as set forth in claim 2, wherein the one or more substances include ceramic beads, carbon and oil control tablets.

10. The shower therapy device as set forth in claim 2, wherein the one or more substances include far-infrared molecules, carbon, and an anti-frizz formula for treating frizzed hair.

11. The shower therapy device as set forth in claim 10, wherein the carbon is a ceramic bead and the far-infrared molecules include Quartz and Hematite.

12. The shower therapy device as set forth in claim 3, wherein the filtering unit includes:
a layer of carbon, calcium sulfite and KDF 85; and
a crystal quartz layer on top of the layer of carbon.

13. The shower therapy device as set forth in claim 3, further comprising hair and skin pod cover having a mesh screen positioned between the hair and skin pod and the filtering unit, the mesh screen allowing for passage of the unfiltered water from the hair and skin pod to the filtering unit.
